(19) United States
(12) Patent Application Publication
(10) Pub. No.: US 2007/0166382 A1
(43) Pub. Date: Jul. 19, 2007

(54) Bioresponsive polymer system for delivery of microbicides

(76) Inventors: Patrick F. Kiser, Salt Lake City, UT (US); David F. Katz, Chapel Hill, NC (US); Russell J. Stewart, Salt Lake City, UT (US)

Correspondence Address: THE MCCALLUM LAW FIRM, P. C., 685 BRIGGS STREET, PO BOX 929, ERIE, CO, 80516, US

(21) Appl. No.: 10/594,196

(22) PCT Filed: Mar. 28, 2005
(86) PCT No.: PCT/US05/10285

§ 371 Date: Sep. 25, 2006

Related U.S. Application Data
(60) Provisional application No. 60/556,796, filed on Mar. 26, 2004.

(51) Int. Cl. A61K 9/14; A61K 31/765

(52) U.S. Cl. 424/486; 424/78.38

(57) ABSTRACT

The polymer systems of the present invention degrade in the presence of an ejaculate. They may further provide degradable sequences that degrade upon contact with an ejaculate and/or microbicides. The polymer systems of the present invention are of use in the oral, rectal or vaginal cavities of an individual for such purposes as the treatment or prevention of sexually transmitted disease, the prevention or promotion of fertility or for hormone replacement therapy.

### CROSS REFERENCE TO RELATED APPLICATIONS

This application claims priority from U.S. Provisional Patent Application No. 60/556,796 filed Mar. 26, 2004.

### BACKGROUND OF THE INVENTION

1. Field of the Invention

The present invention provides compositions and methods for a bioresponsive polymer system capable of an alteration upon contact with an ejaculate. The polymer system of the present invention may further provide microbicides and function as a delivery mechanism for placement of agents in the oral, vaginal or rectal cavities. Such polymer systems may be useful in the prevention or treatment of sexually transmitted diseases, promotion or prevention of fertility, or for hormone replacement therapy.

2. Description of the Related Art

Approximately 65 million people are currently infected with an incurable sexually transmitted disease (STD) in the United States with 15 million new cases reported each year. STDs are difficult to track as many of those with infections remain undiagnosed and are never reported. The most common STDs are Chlamydia, gonorrhea, syphilis, genital herpes, human papillomavirus (HPV), hepatitis B, trichomoniasis, HIV and AIDs.

Currently, there are approximately 40 million people worldwide living with HIV or AIDS, and new diagnoses are occurring at a rate of approximately 12% per year. There is currently no cure for HIV and research into methods of preventing or curing an infection is complicated by ongoing mutations of the viral DNA itself. Therefore, vaccinations currently under development may only protect the population from a small fraction of HIV strains due to the rapid mutation rate of the virus.

Microbicides are topical chemical agents that can block sexually transmitted diseases, including HIV. Referred to as “chemical condoms”, they are formulated into gels, creams, foams, impregnated sponges, suppositories, or films for insertion into the vagina or rectum prior to intercourse. However, use of currently available microbicides is not without risk as they have been shown to make the user more vulnerable to infection by damaging the protective oral, vaginal or anal epithelial layer thereby leaving the infection-prone lower layers exposed. Additionally, current microbicide formulations do not promote retention of the microbide itself in the vagina or rectum.

The development of a delivery system capable of responding to the unique biological environment of the oral, vaginal or anal cavities is needed whereby maintenance of the epithelial layer is maintained, while also promoting retention of the microbicide once applied. Such a formulation would provide an improved method of delivering microbicides in order to prevent sexually transmitted diseases.

### SUMMARY OF THE INVENTION

In accordance with the purpose(s) of this invention, as embodied and broadly described herein, this invention, in one aspect, relates to a polymer system that demonstrates an alteration upon exposure to an ejaculate. Such alteration may be a change in viscosity or modulus, for example, upon exposure to an ejaculate. The polymer system may further provide microbicides which are released upon exposure of the polymer system to an ejaculate. In particular embodiments, the components of an ejaculate that may induce a physical, chemical or enzymatic change in the polymer system include ions, sugars, surfactants, proteolytic and other enzymes and the like. These components and in particular enzymes can be used to induce a reduction in viscosity or modulus in a polymer system. In particular embodiments, the gel may change from a cream-like material to a soluble (sol) polymer system while in other embodiments it may change from a hydrogel-like material to a sol polymer system. In a particular embodiment, microbicides are conjugated to the polymer system. The polymer system of the present invention can be utilized as a method of delivering microbicides, such as for the prevention of sexually transmitted diseases, the prevention or promotion of fertility, replacement of hormones and the like.

### DETAILED DESCRIPTION

The present invention may be understood more readily by reference to the following detailed description of particular embodiments of the invention and Examples included therein.

Particular advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

Before the present invention and/or methods are disclosed and described, it is to be understood that this invention is not limited to specific reagents or synthetic procedures, as such may, of course, vary, unless it is otherwise indicated. It is also to be understood that the terminology used herein is for the purpose of describing particular embodiments only and is not intended to be limiting.

### FIGURES

The following Figures and Tables form part of the present specification and are included to further demonstrate certain embodiments. These embodiments may be better understood by reference to one or more of these Figures and Tables in combination with the detailed description of specific embodiments presented herein.

Table 1 illustrates the change in rheological properties of the individual components of a two polymer system, as well as the resulting polymer system. A higher viscosity gel was created upon mixing the two individual polymers together.

Table 2 illustrates the change in rheological properties of the polymer system in response to exposure to an ejaculate.

FIG. 1 illustrates three illustrative embodiments of the present invention. FIG. 1(a) illustrates a linear chain degradable polymer system according to one embodiment of the present invention wherein (A) is a degradable sequence, (B) is a polymer filament, (C) is a component in an ejaculate which cleaves the degradable sequence, (D) is a remaining moiety resulting from cleavage of the polymer backbone and (E) is also a remaining moiety resulting from cleavage of the polymer backbone.

FIG. 1(b) illustrates a linear chain degradable polymer system made with variable blocks of polymer filaments wherein (A) is a water soluble polymer filament, (B) is a degradable sequence, (C) is a water insoluble polymer filament, (D) is a water soluble polymer filament, (G) is a component in an ejaculate which cleaves the degradable sequence, (F) is a remaining moiety resulting from cleavage of the polymer backbone and (E) is another remaining moiety resulting from cleavage of the polymer backbone according to one embodiment of the present invention.

FIG. 1(c) illustrates degradation of covalent, hydrogen, or ionic bonds which form crosslinks between polymer chains according to an embodiment of the present invention. In this particular embodiment, (A) is polymer component 1, (B) is a degradable sequence, (C) is cross-linking moiety 1, (D) is cross-linking moiety 2, (E) is polymer component 2, (F) is a component in an ejaculate that interacts with (B) and cleaves it into two parts (G) and (H).

FIG. 2 illustrates an interpenetrating polymer network according to one embodiment of the present invention. In this illustration, (A) is a water soluble polymer filament 1 containing cross-linking moieties (D) which allow polymer filament (A) to independently form micelles (C). (B) is a water soluble polymer filament 2, which also contains cross-linked moieties containing degradable sequences. (B) forms micelles (C), which may be formed by cross-linking moieties (D) which are the same as or different from the cross-linking moieties of polymer filament (A). A mixture of (A) and (B) forms an interpenetrating network gel. The viscosity of the gel is reduced when the cross-linking moieties (D) in the micelles (C) are degraded.

FIG. 3 illustrates three particular embodiments of the present invention. FIG. 3(a) illustrates a self-associated degradable polymer system in accordance with an embodiment of the present invention. In this illustration, polymer 1 contains degradable sequences (B) and micelle forming hydrophobic chains (C). In the presence of an ejaculate containing component (D), degradable sequences (B) are cleaved into fragments (F) and (G). Polymer (A), comprising fragment (F), and hydrophobic micelle chain (E), comprising fragment (G), are thereby severed.

FIG. 3(b) illustrates the displacement of an interaction between two chains by a component in an ejaculate according to an embodiment of the present invention. This figure illustrates polymer components (A) and (E) interacting via moieties (B) and (C) to form a temporary crosslink. In the presence of an ejaculate including component (D), (B) is displaced by (D) and the crosslinks are broken between polymer (A) and (E).

FIG. 3(c) illustrates the degradation of a crosslinker by a component in an ejaculate according to an embodiment of the present invention. The figure illustrates polymer component 1 (A) interacting through crosslinks to polymer component 2 (E). In the presence of an ejaculate which contains component (F) the crosslinks are disrupted between polymer 1 and 2.

FIG. 4 illustrates particular polymer systems of the present invention. FIG. 4(a) illustrates another mechanism of degrading a crosslinker by a component in an ejaculate according to a particular embodiment of the present invention. In this instance, polymer 1(A) and polymer 2 (F) are crosslinked by ionic interactions (C) between polymer-bound moieties (D) and ionic components (S). In the presence of an ejaculate, a component of which is an ionic complexing agent (E), the crosslink is broken through competition or blocking by (E) for ionic component S.

FIG. 4 (b) illustrates degradation of ionically crosslinked polymers according to an embodiment of the present invention. In this instance, polymer 1 (A) interacts with polymer 2 (F) via ionic interactions between opposing groups (B and D) on each polymer. The addition of an ejaculate which includes component (E) disrupts these ionic interactions and breaks the ionic bonds.

### DEFINITIONS

For the purposes of the present invention, the following terms shall have the following meanings:

For purposes of the present invention, the term, “microbicide” will refer to any agent that prevents, treats, inactivates, degrades or in any other way affects a causal agent of a sexually transmitted disease. Examples of such agents include antiviral drugs, traditional microbicides that destroy microbes, such as viruses and bacteria, and the like. Additionally, the term will further include any agent that prevents or promotes fertility. Such agents may be useful in in-vitro fertilization procedures, as a family planning methodology or as a way to supplement a particular hormone or combination of hormones in an individual.

Moreover, for the purposes of the present invention, the term “a” or “an” entity refers to one or more of that entity, for example, “a protein” or “an enzyme” refers to one or more of those elements or at least one element. As such, the terms “a” and “an”, “one or more” and “at least one” can be used interchangeably herein. It is also to be noted that the terms “comprising”, “including”, and “having” can be used interchangeably. Furthermore, “selected from the group consisting of” refers to one or more of the elements in the list that follows, including mixtures (i.e. combinations) of two or more of the elements.

For the purposes of the present invention, ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about”, it will be understood that the particular value forms another embodiment. It will be further understood that the endpoints of each of the ranges are significant both in relation to the other endpoint, and independently of the other endpoint.

Reference will now be made in detail to particular embodiments of the invention.

### Polymers

The polymer systems of the present invention are bioresponsive to the oral, rectal or vaginal cavity in which they are applied upon exposure to an ejaculate. In response to exposure to an ejaculate, such polymer systems experience an alteration in viscosity or modulus, for example. Any polymer known in the art may be used in the present invention.

The polymers of use in the present invention, include but are not limited to, the class of water soluble synthetic polymers, such as ethylene glycol, poly(ethylene) glycol, poly(ethylene oxide), poly(vinylpyrolidone), poly(ethylene oxide)-co-poly(propylene oxide), and poly(ethyloxazoline), poly(urethanes), poly(vinyl alcohol), poly(sulfostyrenes), carboxymethylcellulose, cellulose acetate, modified celluloses, cellulose acetate phthalate, soluble derivatives of cellulose acetate phthalate, dextran, nylons, carboxymethylcellulose and carbopols and their copolymers graft comb polymers and derivatives. Additionally, the class of water soluble polymers include the water soluble natural polymers, including but not limited to, poly(saccharides), proteins, poly(aminoacids) alginates, chondroitin sulphate, caarageenans, chitosan, heparin, hyaluronic acid, deoxyribonucleic acid, poly(aminoacids) and other sugar containing polymers and their copolymers and derivatives.

In another embodiment of the present invention, polymers include acrylic and acrylate based polymers which are formed from acrylic and acrylate based monomers, which include, but are not limited to, 2-hydroxypropylmethacrylamide, 2-hydroxyethylacrylate, acrylic acid, methacrylic acid and other similar monomers. Additionally, co-polymers, block copolymers and their derivatives may be used in the present invention and may be formed by free radical, anionic or cationic polymerization, ring opening metathesis polymerization, and other known methods.

In another embodiment of the present invention, hydrophobic degradable polymers and their oligomers may be used as components in the polymer system as long as the required water solubility is not compromised. Polymers of this type include, but are not limited to, the poly(esters), poly(ethers), poly(caprolactone), poly(valerolactone), poly(α-hydroxyesters) and their copolymers and derivatives.

In another embodiment the polymer system may be composed of self-assembling amphiphilic monomers which have at one end a water soluble degradable sequence, in the middle portion a hydrogen bonding sequence composed of peptides terminated in a hydrophobic chain. These amphiphilic monomers are known to those skilled in the art to assemble into long fibers which form a gel structure. In a particular embodiment the degradable sequence is composed of charged peptide substrates of prostrate specific antigen, the middle sequence is composed of peptides composed of alanine and Glycine and the hydrophobic chain is composed of an alkyl tail of 12 to 24 carbons.

In another particular embodiment, the polymers are water soluble resulting in cross-linked polymers, such as in a hydrogel or high viscosity cream. In another embodiment the polymer system may be composed of monomers or polymer filaments that contain negatively charged moieties. These negatively charged moieties include sulfate, and carboxylate moieties.

In another particular embodiment, the polymer system is described as being composed of two distinct polymers which form the polymer system. In this case the two distinct polymers may be of the same chemical class of polymers or different classes.

In particular embodiments, the polymer is a preformed polymer which is then suitably modified. Modifications, including polymerization with a wide variety of described functionalities, are well known in the art.

In one embodiment of the present invention, the polymer system is composed of two distinct low viscosity polymers. Upon mixing the two polymers, a gel forms due to the formation of interactions between the two polymers. In a particular embodiment, such interaction is a crosslink. These interactions may be temporarily disrupted under mechanical or sheer stresses, which may allow sheer thinning. Additionally, upon exposure to an ejaculate, the interactions may be degraded or destroyed by a component in the ejaculate creating a low viscosity fluid.

The polymer systems of the present invention may further include microbicides. The polymer systems may be optimized for the functional requirements of a particular microbicide associated with a particular polymer system. For example, polymer systems of the present invention can be produced that respond to the physical forces inherent in intercourse. In a particular embodiment, the polymer systems of the present invention containing microbicides can be engineered or formulated to exhibit specific rheological characteristics such as the existence of yield stresses and sheer thinning. The presence of yield stresses may aid in retention of the polymer system in the oral, vaginal or anal cavity prior to intercourse. Sheer thinning may also promote the ability of the material to be spread before and during intercourse. One skilled in the art will know how to utilize a particular microbicide's rheological, adhesive and diffusive properties in order to respond to physical changes present in the vagina upon exposure to an ejaculate.

Changes in the environment, such as the addition of seminal proteases or alterations in pH, can be predicted in order to enable and enhance different phases of microbicide deployment and delivery. In a particular embodiment, a liquid form of the polymer system may be desired for ease of application, thereby promoting penetration during intercourse, ease of use and coating of the oral, vaginal or anal cavity. In another particular embodiment, it may be desirable for the polymer system to gel promoting coating, retention and decreased bioavailability of the microbicide. In another particular embodiment, upon contact with an ejaculate the polymer system may undergo liquefaction and release the microbicides which can be later removed by gravity or other forces from the body. In another exemplary embodiment, it may be desirable for a molecular layer of polymer to be left behind to provide a continued level of protection to the tissue. One skilled in the art understands how to use the inherent characteristics of particular polymers and microbicides to create the polymer systems containing microbicides of the present invention.

The polymer system of the present invention can be applied anywhere. In a particular embodiment, it is applied to an oral, rectal or vaginal cavity.

### Degradable Sequences

The polymer systems of the present invention degrade in the presence of an ejaculate. In a particular embodiment, a degradable sequence may be utilized that is susceptible to degradation upon contact with an ejaculate. The components of an ejaculate that may be involved in degradation of polymer systems of the present invention include, but are not limited to, protein, carbohydrates, phospholipids, albumin, citrate, sodium, fructose, choline, chloride, glycerol phosphocholine, sialic acid, glucose, lactoferrin, potassium, spermine, phosphate, triglicerides, lactic acid, inositol, urea, cholesterol, glutantione, calcium, camitine, creatine, pyruvic acid, zinc, ascorbic acid, magnesium, glutamic acid, sorbitol, lipid phosphatases, uric acid, transferring, creatinine, ammonia, prostate specific antigen (PSA) and semenogellin, for example. Many enzymes are also present in an ejaculate and include Alanyl aminopeptidase (AAPS), alanyl aminopeptidase (Ap N), granulocyte elastase enolase, angiotensin converting enzyme (ACE), dipeptidylpeptidase IV, kallikrein hK2 (Kininogenase), Gastricsin, matrix metalloproteinases (MMP-2 and MMP-9), Kallikrein hK3 and the like. The substrates of these enzymes or other components of an ejaculate are well known in the art allowing for the creation of the degradable polymer systems of the present invention. Any component of an ejaculate may be utilized to degrade the polymer system of the present invention.

In one embodiment, degradable sequences are those that are susceptible to chemical, physical or enzymatic degradation. Chemical degradation is largely isolated to functional groups which are likely stable in the natural pH of the vagina of approximately 4-5 while becoming unstable in the presence of a higher pH, such as 7.5 which is found in an ejaculate. Chemical functionalities that fit this description include, but are not limited to, esters, such as oligomers of the alpha-hydroxyesters, amides, imides, and the like. Degradable sequences may be chemically cleaved by acids, bases, alcohols, and chelating agents, for example. In a particular embodiment, the degradable sequences are oligomers of alpha-hydroxyesters that degrade rapidly via base-promoted hydrolysis, where the base is a part of an ejaculate. Oligomers of N=2 to 6 of glycolic acid esters are included in this embodiment.

Degradable sequences may alternatively be degraded and therefore affected by physical means, such as changes in pH, ionic strength, temperature, sheer stress and pressure, for example. Physical means may further provide for forces exerted during intercourse itself, such as sheer stress.

Degradation may also occur via proteolytic enzymes in an ejaculate. One such enzyme, PSA, is capable of causing degradation of polymer systems of the present invention. Degradation may also be triggered by low levels of proteolyic enzymes found in an ejaculate, such as peptidases and hyaluronidases, which may further act to trigger changes in the viscosity of the gel. In a particular embodiment where hyaluronidases are utilized to trigger a degradation sequence, a hyaluronic acid based polymer or a polymer containing sub-units of hyaluronic acid would be utilized. Substrates for proteolytic enzymes are well known in the art.

In particular modes of the invention it may be desirable to take a suitably protected or unprotected degradable sequence and produce a reactive conjugate to attach it within or to one or more of the polymers of the polymer system in order to construct a suitable architecture for the polymer system. This material is referred to as a degradable sequence conjugate (DSC). In some cases the terminating functional groups for the DSC will be the same or different depending on the polymer architecture of the polymer system and the requirements of the mode of the invention.

Other enzymes found in an ejaculate that have the ability to cause degradation of the polymer systems of the present invention include but are not limited to alpha and beta glucosidase, lysophospholipases, lysozyme, mannosidases, pepsinogen I, pepsinogen II, pepsinogen III, phospholipase and the like.

### Creation of Polymer System

Polymer systems containing degradable sequences susceptible to degradation upon exposure to an ejaculate of the present invention may be made by any method known in the art.

In one embodiment, the polymer system will gel via physical or chemical interactions between two components, in which each polymer component alone will not gel but mixing of the multiple polymer components results in formation of a gel. In another embodiment, sugar specific mucoadhesive moieties can be included in the polymer backbone which will promote coating to the epithelium of the oral, vaginal or rectal mucosa and also may bind to components of causative agents of sexually transmitted diseases, such as HIV glycoproteins.

In another embodiment of the present invention a suitably functionalized degradable sequence with two reactive end groups is created which can be incorporated into a polymer system by copolymerization to create a crosslinked structure held together by degradable sequences.

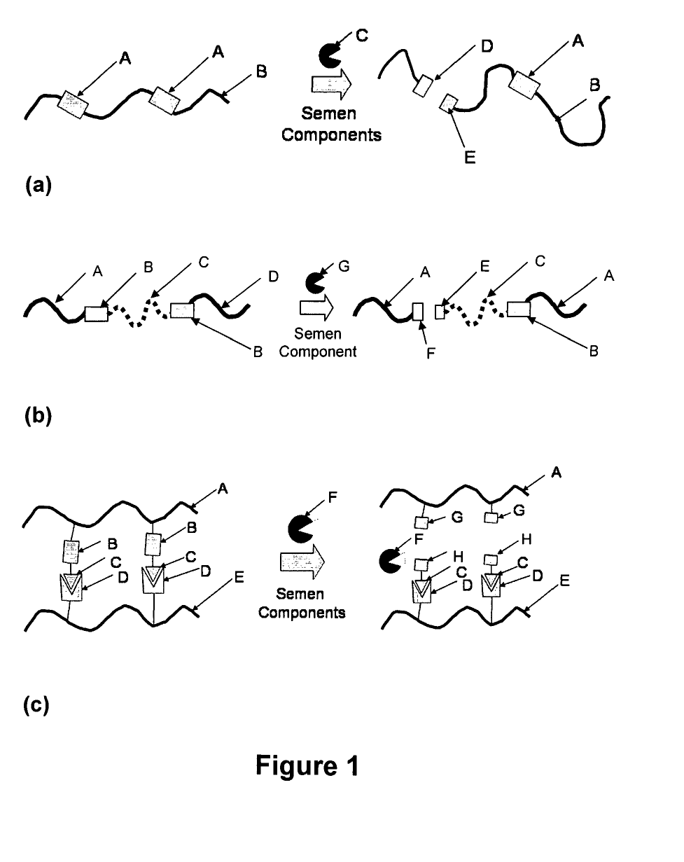

In another embodiment a polymer system may be created by placing a degradable sequence between segments of the polymer filament or by linking together polymer filaments into a higher molecular weight structure (FIG. 1(a)). Additionally, water soluble linear prepolymer filaments can be copolymerized into a higher molecular weight linear structure with degradable sequences between the prepolymer segments. Alternatively, the α-end functional group of the polymer filament which contains the degradable sequence can be polymerized with the ω functional group of the same type of polymer filament generating a high molecular weight structure. In a particular embodiment, the linear prepolymer filament is poly(ethylene glycol). Other water soluble synthetic polymer filaments and water soluble natural polymer filaments, such as suitably functionalized end group telechelic polymers, can also be used and the components (A) and (B) in FIG. 1(a) can be assembled using suitable linking chemistry known to those skilled in the art. Telechelic polymers of the present invention may be selected from the group selected from poly(ethylene oxide), polypropylene oxide, block copolymers of polyethylene oxide, polypropylene oxide and the like. Many other reactive end group chemistries, such as this one, may be used in the present invention and are known to those skilled in this art.

In a particular embodiment, the degradable sequence (A) illustrated in FIG. 1(a) is a peptide or sugar that is cleavable by proteases or other enzymes in an ejaculate.

In another particular embodiment, a telechelic α-hydroxy and ω-carboxylic acid pre-polymer containing the degradable sequence on one end can be constructed which is then polymerized with identical pre-polymer fragments or a similar polymer using standard condensation conditions in order to construct degradable high molecular weight architecture.

In another embodiment, the degradable sequence (A) in FIG. 1(a) may have a homo-bifunctional reactive group at both ends of the degradable sequence which will react with a suitably functionalized α,ω-telechelic polymer much like the manner of a urethane. In this embodiment a diisocyanate degradable sequence conjugate where the degradable sequence sits between the isocyanate reactive groups can be condensed with a α,ω-diol using methods known to those skilled in the art. Many other reactive group chemistries, such as this one, may be used in the present invention and are known to those skilled in this art.

In another embodiment soluble proteins are assembled with degradable sequences interspersed within a sequence of synthetic amino acids similar to semenogelins involved in formation of the crosslinked seminal collagulum.

Polymers (B) of use in FIG. 1(a) with the present invention include, but are not limited to, poly(aminoacids), ethylene glycol oligomer, poly(ethylene) glycol, poly(ethylene oxide), poly(vinylpyrolidone), poly(ethylene oxide)-co-poly(propylene oxide), poly(ethyloxazoline), dextran, poly(vinylpyrolidone), nylons and urethanes, and their copolymers and derivatives with a plurality of degradable sequences interspersed along the chain.

In an additional embodiment, one may use a hyaluronic acid gel or a hyaluronic acid conjugated with hydrophobic groups or water soluble polymer filaments in the form of a graft comb polymer, where the degradable sequence (A) of FIG. 1(a) is naturally incorporated in the polymer backbone. In this embodiment the polymer would be degraded by hyaluronidase in the ejaculate into a lower molecular weight polymer with a lower viscosity.

FIG. 1(c) illustrates a linear chain degradable polymer system made with variable blocks of polymer filaments. In this embodiment, degradable sequences (B) are attached between blocks of polymer filaments (A, C, D) in ABCBD type block co-polymer fashion. Here a degradable sequence is inserted between the (A) and (C) polymer filaments and the (C) and (D) polymer filaments in FIG. 1(b) forming a triblock polymer with two degradable sequences (B). (A) and (D) can be comprised of polymer filaments from the class of water soluble polymers, and the (C) block can be comprised of a water insoluble polymer filament. Alternatively, all polymer filaments in FIG. 1(b) can be composed of polymer filaments from the class of water soluble polymers. Additionally, (A) and (D) can comprised of polymer filaments from the class of water insoluble polymers and the (D) block can be comprised of a water soluble polymer filament.

It will be understood by those skilled in the art that the embodiment of FIG. 1(b) may be synthesized with varying numbers of polymer filaments or degradable sequences. In a particular embodiment, the degradable sequence (B) is a peptide or sugar that is cleavable by proteases or other enzymes in an ejaculate.

In a particular embodiment, mono-functional polymer filaments, for example (A) and (D) of FIG. 1(b), would be end-capped with suitably functionalized degradable sequences (B). Two of these polymeric molecules can then be reacted with an α,ω-telechelic polymer (C) to form the ABCBD architecture. Alternatively, the (C) polymer filament can be capped at both ends and this could be reacted with suitably functionalized (A) and (D) polymer filaments to form the ABCBD architecture.

In another particular embodiment, the (A) and (D) blocks of FIG. 1(b) are mono reactive poly(ethylene oxide) and the (C) block is a α,ω-diol poly-propylene oxide or poly(ethylene oxide). To synthesize these compounds, one can conjugate (A) and (D) to a degradable sequence. Molecules of this degradable sequence conjugate bound to (A) and (D) can then be reacted with a suitably α,ω-functionalized poly(propylene oxide) block to form the polymer system.

In another particular embodiment, an α,ω-telechelic diol poly(propylene oxide) block water insoluble polymer filament is reacted with a carboxy terminated degradable sequence conjugate which is attached to polymer filament (A) of FIG. 1(b), where (A) is poly(ethylene oxide).

In another particular embodiment, an α,ω-telechelic diamine of a water insoluble polymer filament is used for the polymer filament (C) of FIG. 1(b), and is reacted with a carboxylic acid terminus of a peptide degradable sequence conjugate to form a bis-functionalized polymeric degradable sequence conjugate of filament (C), which is then reacted with a suitably functionalized polymer filament (A) or (D) to form the polymer system.

In another particular embodiment, a α,ω-telechelic polymer filament (C) of FIG. 1(b) diacid block is reacted with the N terminus of a peptide degradable sequence conjugate to form a bis-functionalized polymeric degradable sequence conjugate, which is then reacted with a suitably functionalized polymer filament (A) and/or (D).

In another embodiment, an α,ω-telechelic polymer filament diacid (C) block could be reacted with a hydroxyl functionalized degradable sequence conjugate containing the (A) and/or (D) block.

In another embodiment, the polymers that are suitable for the FIG. 1(b) filaments (A) and (D) are end functionalized water soluble polymers including, thiol terminated 2-hydroxypropyl methacrylamide, thiol terminated hydroxyethyl methacrylate and other end functionalized acrylate polymers. Included in the hydrophobic (C) block are polymers such as poly(esters), poly(saccharides), poly(propylene oxide), poly(carbonates) and other non-water soluble polymers.

FIG. 1(c) illustrates the degradation of covalent, hydrogen, or ionic bonding crosslinks between polymer chains. In this embodiment, a polymer filament (A) is constructed in such a way that it is functionalized with at least one degradable sequence conjugate terminated with at least one bonding moiety (C) that can interact through covalent, hydrogen, and/or ionic bonding with a complimentary bonding moiety (D) on another polymer filament (E) of the polymer system. When exposed to the appropriate component in an ejaculate the degradable sequence(s) (B) will be cleaved and the viscosity or modulus of the polymer system will then be reduced.

In FIG. 1(c), (A) and (E) may be the same or different polymer filaments. In a particular embodiment, polymer filaments (A) and (E) are water soluble natural polymers or water soluble synthetic polymer filaments. In another particular embodiment, the degradable sequence (B) is a peptide or sugar capable of cleavage by proteases or enzymes in an ejaculate. The (C to D) connection shown in FIG. 1(c) can be made through hydrogen bonding interactions based suitable hydrogen bond donor and acceptor pairs. In one embodiment the hydrogen bond donor acceptor pair are cyanuric acid and melamine. Other hydrogen bonding constructs are known to those skilled in the art.

In another particular embodiment, the (C to D) interaction of FIG. 1(c) is covalent in nature and involves the use of carbon-carbon, carbon-oxygen, carbon-sulfur, sulfur-sulfur or carbon-nitrogen bonds to link the filaments (A) and (E) together via suitable linking chemistry. In a particular embodiment, the degradable sequence (B) is terminated in a thiol and the complimentary bonding moiety (D) contains a Michael acceptor such as an α,β-unsaturated ester or ketone, a vinylic sulfone, or another suitable Michael acceptor. When the thiol is mixed with the Michael acceptor, crosslinking will occur and a higher molecular weight structure will be produced.

In another particular embodiment, polymer filament (A) of FIG. 1(c) is a water soluble polymer and degradable sequence (B) is a sequence made up of a peptide susceptible to proteases contained within an ejaculate which is attached to (A) through suitable reactive groups including thiol, alcohol, amine, carboxylic acid carbonate, carbamate, hydrazone, hydrazine, aldehyde, cyclic ether, acid halide, acyl azide, succinimidyl ester, imidazolide or amino functionality.

In another embodiment, polymer filament (A) of FIG. 1(c) would have only one attachment site for degradable sequence (B) and a plurality of filaments (A) would be attached to polymer filament (E) with a plurality of complimentary bonding moieties (D). Alternatively, in another embodiment, polymer filament (E) would have only one attachment site for the complimentary bonding moiety (D) and a plurality of filaments (E) would be attached to polymer filament (A) with a plurality of bonding moieties (C). Both of these embodiments will result in graft comb polymers.

The backbone structure for polymer filament (A) and polymer filament (E) of FIG. 1(c) may be the same although they will be functionalized differently with degradable sequences (B), and bonding moieties (C) and (D) components. Furthermore, it will understood by one skilled in the art that additional polymer filaments (not shown) may similarly interact with either polymer filament (A) or (E), thus forming a layered polymer system.

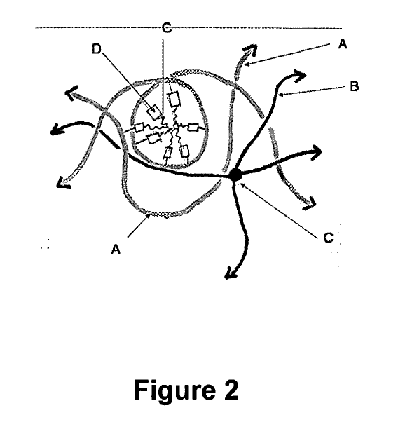

FIG. 2 illustrates an interpenetrating polymer network containing a water soluble polymer filament (A), which forms hydrophobic micelles (C) through intrapolymer interactions (E). Degradable segments (D) connect polymer filament (A) to interacting moieties (E). A second polymer filament (B), also containing intrapolymer micelles (C), forms an interpenetrating polymer network.

In this particular embodiment, two or more polymers are highly viscous when not mixed but form a gel when mixed together, through the formation of an interpenetrating network. By placing a degradable sequence (D) between one of the interacting moieties (E) and the polymer filament (A) a reduction in viscosity results when the degradable sequence (D) interacts with the appropriate component in an ejaculate. By breaking the micelle interactions (E) of polymer (A) and/or (B) the degree of crosslinking of the gel is changed.

In a particular embodiment, the degradable sequence (D) of FIG. 1(a) is a peptide capable of cleavage by proteases in an ejaculate.

In a particular embodiment, a hydrolytically labile degradable sequence (D) as shown in FIG. 1(a) is utilized to cause a reduction in the viscosity of the polymer system. In order to accomplish such a viscosity change, one creates a degradable oligo-alpha-hydroxy ester which is terminated with a hydrophobic group (see below, compound 2). This hydrolytically labile oligo-alpha-hydroxy ester can then be conjugated to a polymerizable moiety and co-polymerized with a water soluble monomer in a ratio of 1 to 50 mole percent. A particular embodiment comprises 7 mole percent of the oligo-alpha-hydroxy ester with the water soluble monomer 2-hydroxypropylmethacrylamide or 2-methacroylethyphosphocholine to form polymer (A) of FIG. 1(a). The resulting polymer (A) can then be mixed with another suitably functionalized polymer filament (B). Polymer filament (B) may be similarly constructed to contain intrapolymer micelles (C). A mixture of (A) and (B) creates a gel which is stable for days to months at pH ˜4 (the normal pH of the vagina).

In a particular embodiment, polymer (B) of FIG. 1(a) is a water soluble zwitterionic polymer containing carboxylic acid groups. When this gel is incubated at pH 7.4 (the pH of semen), the gel network structure can be broken down over several hours by hydrolysis of the oligo-ester crosslinking moieties (D). If the length of the oligo ester is increased, the gel will exhibit reduced viscosity at a more rapid rate because of increased ester hydrolysis. However, in other embodiments, different water soluble monomers can be used for this component such as methacryloyl-phosphocholine based polymers copolymerized with monomers containing carboxylic acid functionalities and other degradable moieties known to those skilled in the art.

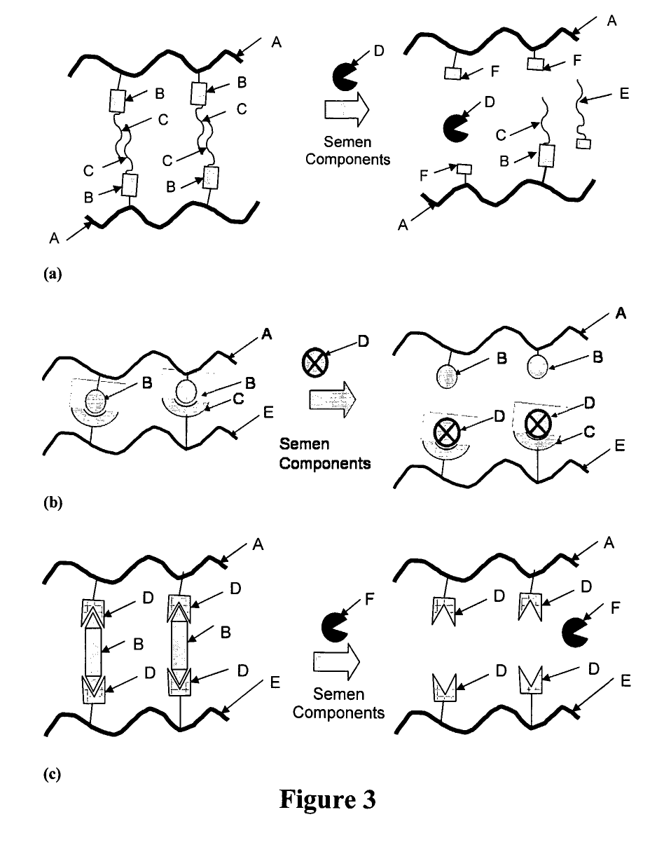

FIG. 3(a) illustrates a self-associated degradable polymer system. In this embodiment degradable sequence conjugates (B) are attached to a polymer (A) by a conjugation technique well known in the art. A hydrophobic group (C) is tethered to degradable sequence (B). Suitable hydrophobic groups include those with a plurality of carbon atoms including but not limited to 4 to 18 carbon atoms depending on the polymer filament (A) or the nature of the degradable sequence (B) itself. When this material is subjected to the appropriate component in an ejaculate the degradable sequence will be cleaved into fragments (F) and (G) and the polymer will experience a reduction in viscosity or modulus.

In a particular embodiment, the degradable sequence (B) of FIG. 3(a) is a peptide or sugar capable of cleavage by a protease or enzyme in an ejaculate. In another particular embodiment, a peptide degradable sequence with or without a PEG spacer is conjugated to a water soluble synthetic polymer filament or a water soluble natural polymer filament (A). In a particular embodiment, polymer filament (A) is chitosan. In another particular embodiment, the polymer filament (A) is a poly(acrylic acid)-graft-poly(ethylene oxide) graft comb polymer where the poly(ethylene oxide) graft is terminated in a hydrophobic group and the degradable sequence (B) sits between either the polymer filament (A) or the terminus of the poly(ethylene oxide) and the hydrophobic group (C).

FIG. 3(b) illustrates the conjugation of a moiety (B) to one polymer filament (A) and the conjugation of another moiety (C) to polymer filament (E). Moiety (C) binds moiety (B). When the polymer system comes in contact with the components in an ejaculate (D), one of the components (D) preferentially binds to (C) and displaces (B). The crosslinks are broken resulting in a lower viscosity polymer system or lower modulus polymer gel. In this mode of the invention (A) and (E) may be the same or different polymer filaments.

In a particular embodiment, polymer filament (A) of FIG. 3(b) is selected from the class of water soluble natural and synthetic polymer filaments and to this polymer filament (A) is attached a sugar moiety containing a 1,2 diol (B). In a particular embodiment, polymers (A) and (E) come from but are not limited to the set of approved polymers for human use such poly(acrylic acid) and poly(hydroxypropylmethacrylamide). Polymer filament (E) is a member of the class of water soluble natural and synthetic polymer filaments as well. To filament (E) are conjugated boronic acid moieties (C). When (A/B) and (E/C) are mixed, a boronic acid ester will form and the material will form a higher molecular weight gel or higher viscosity material. When this material comes in contact with an ejaculate, sugars (D) present within the ejaculate will displace the interaction between (B) and (C) and result in a lower viscosity or lower modulus material.

FIG. 3(c) illustrates the degradation of crosslinking in a polymer system after exposure to an ejaculate. In this embodiment a crosslinking component (B) acts as a crosslinker or gelling agent between two polymer filaments (A) and (E) containing moieties (D) which interact with (B) through covalent, ionic, hydrogen, electrostatic or van der Waals forces to form a higher molecular weight network structure. When exposed to an ejaculate, the crosslinking moiety (B) loses contact with the interacting moieties (D) on the polymers (A) and (E). In this mode of the invention (A) and (E) may be the same or different polymer filaments drawing from the classes of water soluble natural and synthetic polymer filaments.

In a particular embodiment of FIG. 3(c), the polymer filaments (A) and (E) are chitosan, the crosslinking component (B) is 2-phospoglycerate and the ejaculate component (F) is selected from the group consisting of granulocyte elastase or enolase which metabolizes 2-phosphoglycerate.

In another particular embodiment of FIG. 3(c), (B) is a crosslinking degradable segment with cationic or anionic groups attached to the ends. Additionally, in another particular embodiment, if (D) is anionic then (B) is cationic or if (D) contains cationic moieties then (B) would be anionic.

Lastly, in another particular embodiment, (B) of FIG. 3(c) is a crosslinking degradable segment containing degradable peptide or sugar sequences as described above with hydrogen bond donors or acceptors attached to the ends. In another particular embodiment, if (D) is a hydrogen bond donor then (B) contains hydrogen bond acceptors and if (D) contains hydrogen bond acceptors then (B) would contain hydrogen bond donors.

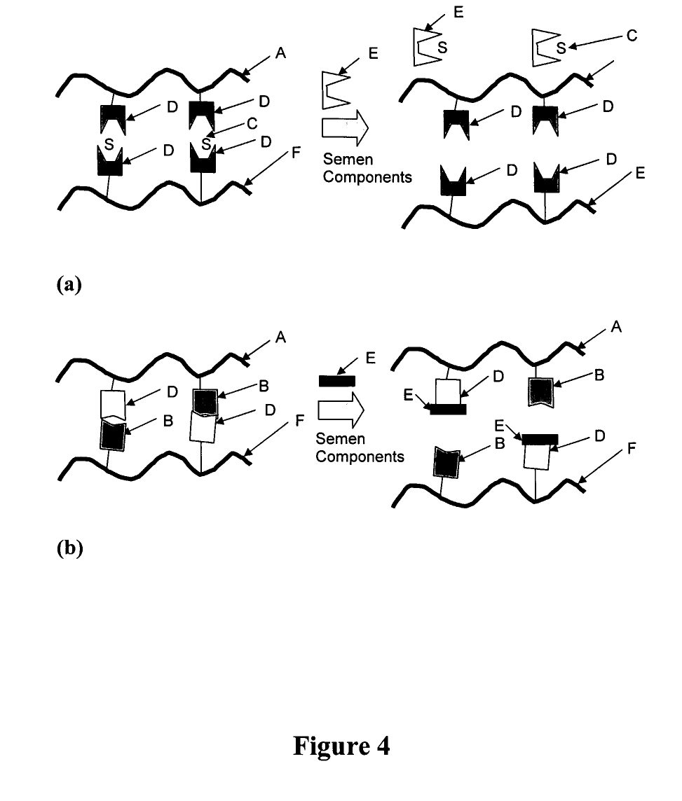

FIG. 4 (a) illustrates another mechanism for degradation of crosslinked moieties. In this embodiment a crosslinking substrate (S) acts as a crosslinker or gelling agent between two polymer filaments each containing moiety (D), which interacts with (S) to form a complex (C) and a higher molecular weight structure. The complex remains intact through, ionic, electrostatic, hydrogen or van der Waals interactions. When mixed with a component of an ejaculate (E), the polymer system is degraded because the ejaculate components (E) interact more strongly with (S) than (D). In this mode of the invention (A) and (F) may be the same or different polymer filaments drawing from the classes of water soluble natural and synthetic polymer filaments.

In a particular embodiment of FIG. 4 (a), (A) and (F) are alginate and (S) is a divalent cation like calcium. Additionally, the ejaculate component (E) is a polyvalent ion chelator, like citrate, succinate or phosphate, which is present in an ejaculate.

In another embodiment, polymer systems of the present invention are ionically cross-linked. In a particular embodiment, two or more distinct polymers interact via ionic interactions between opposing groups on each polymer. FIG. 4(b) illustrates this polymer system wherein polymer 1 (A) interacts with polymer 2 (F) via ionic interactions between opposing groups (B and D) on each polymer. The addition of an ejaculate which includes component (E) disrupts these ionic interactions and breaks the ionic bonds.

In another embodiment water soluble polymer filaments may be crosslinked by a degradable sequence to form a polymer gel. This gel may be placed in the body and upon exposure to an ejaculate the degradable sequence is susceptible to degradation causing the gel to undergo a gel to sol transition. The crosslinked structure may be formed in one or more steps from crosslinking and non-crosslinking monomers. Alternatively, they may be preformed and made suitably reactive in order to react with a suitably functionalized crosslinker containing the degradable sequence. (See example 4). Suitably functionalized crosslinkers and reactive polymer filaments are known to those skilled in the art.

In another embodiment degradable sequences are incorporated into linear or branched polymer filaments and these filaments can then be crosslinked with a degradable sequence or a non-degradable sequence to form a polymer gel. In a particular embodiment, the polymer system of the present invention will experience a gel to sol transition upon exposure to an ejaculate.

In another embodiment polymer systems of the present invention naturally form a physical gel and can be suitably functionalized with pH sensitive degradable groups such that when it is placed in the vaginal cavity at pH 4 the polymer system is a gel. When the pH changes because of the presence of an ejaculate the pH sensitive groups become charged and disrupt the structure of the gel thus causing a gel to sol transition in the polymer system. (see example 2).

In another embodiment the polymer system naturally forms a physical gel can be functionalized with chemically degradable sequence such that when it is placed in the body the polymer system is a gel. When the pH changes because of the presence of an ejaculate the chemically degradable sequence is chemically modified and the resulting polymer system degrades causing a gel to sol transition (see example 3).

In a particular embodiment of the present invention, the polymer system is formed into a microparticle or nanoparticle. The means by which one may form a microparticle or nanoparticle are well known in the art.

### Sexually Transmitted Diseases

Any sexually transmitted disease may be treated with the polymer systems of the present invention. Examples include, but are not limited to, HIV, AIDS, gonorrhea, Chlamydia, trichomonal infections, human papilloma virus (HPV), syphilis, genital herpes, HIV, AIDs and the like.

### Microbicides

Microbicides suitable for use with the present invention include, but are not limited to, entry or fusion inhibitors, nonnucleoside reverse transcriptase inhibitors, nucleoside reverse transcriptase inhibitors, protease inhibitors, detergents, surfactants, anti-metabolites, competitive binding inhibitors and the like.

Entry and fusion inhibitors of the present invention may be selected from the group consisting of, but not limited to, Enfuvirtide (Fuzeon, T-20), AMD11070, PRO542, SCH-C, T-1249, TNX-355, cyanovirin and the like.

Nonnucleoside Reverse Transcriptase Inhibitors of the present invention may be selected from the group consisting of, but not limited to, Delavirdine (Rescriptor), Efavirenz (Sustiva), Nevirapine (Viramune), Calanolide A, Capravirine, Epivir, Hivid, TMC125 and the like.

Nucleoside Reverse Transcriptase Inhibitors of the present invention may be selected from the group consisting of, but not limited to, Abacavir (Ziagen), Abacavir+Lamivudine+Zidovudine (Trizivir), Didanosine (Videx, ddl), Emtricitabine (Emtriva, FTC), Lamivudine (Epivir, eTC), Lamivudine+Zidovudine (Combivir), Stavudine (Zerit, d4t), Tenofovir DF (Viread), Delavirdine (Rescriptor) Zalcitabine (Hivid, ddc), Zidovudine (Retrovir, AZT, ZDR) and the like.

Protease inhibitors of the present invention may be selected from the group consisting of, but not limited to, Amprenavir (Agenerase), Atazanavir (Reyataz), Fosamprenavir (Lexiva, 908), Indinavir (Crixivan), Lopinavir+Ritonavir (Kaletra), Nelfinavir (Viracept), Ritonavir (Norvir), Emtriva, Saquinavir (Fortovase, Invirase), Invirase, Agenerase and the like.

Examples of detergents and surfactants may be selected from the group consisting of, but not limited to, octoxynol-9, chlorhexidine, and benzalkonium chloride and the like. Examples of anti-metabolites of use in the present invention include AZT and the like. Additionally, competitive binding inhibitors, such as dextran, may also be utilized in the present invention.

Microbicides of the present invention that destroy infectious agents may be selected from the group consisting of, but not limited to, viruses, bacteria, prions and the like, include spermicides, such as nonoxynol-9, benzalkonium chloride, C31G, Carbopol 974P, Carrageenan, Cyanovirin-N, fuzeon, hydroxyethyl cellulose, PRO 2000, UC-781, menfegol and the like; inhibitors of viral adsorption, such as dextran sulfate and the like; inhibitors of viral proteases, such as saquinavir and the like; antivirals, such as ribavirin, acyclovir, ganciclovir and the like.

Microbicides of the present invention may also be any agent selected from the group consisting of antibiotics, antifungals, anti-inflammatories, antivirals, antiparasitics, chemotherapeutics, antitoxins, immunotherapeutics, integrase inhibitors and the like.

Microbicides of the present invention that function as birth control agents include, but are not limited to, ethinyl estradiol, norethindrone, levonorgestrel, ethynodiol diacetate, ethynodiol diacetate, RU486, mifepristone, mifegyne, mifeprex and the like.

Microbicides of the present invention that function as hormone replacement agents include, but are not limited to, estrogen, progestin, estrogen and progestin, and the like.

Microbicides of the present invention can be any agent for application to the oral, anal or vaginal cavity.

The polymer systems of the present invention may contain one or more microbicides. The microbicides can be used alone or in combination with any other drug. The present invention includes any combination of polymer system and microbicides.

### EXAMPLES

It should be appreciated by those skilled in the art that the techniques disclosed in the examples which follow represent techniques discovered by the inventors to function well in the practice of the invention, and thus can be considered to constitute particular modes for its practice. However, those of skill in the art should appreciate, in light of the present disclosure, that many changes can be made in the specific embodiments disclosed herein which will still obtain a like or similar result without departing from the spirit and scope of the invention.

### Example 1

### Preparation of Two Component Ejaculate-Degradable Polymer System

This exemplary polymer system is composed of a two component polymer system that gels when mixed together. One of the polymer components contains α-hydroxy acids that are degraded when the system is changed from pH 4 to pH 7 by an ejaculate. This causes the system to undergo a gel to sol transition and show a reduction in viscosity over time in the presence of an ejaculate.

### Synthesis of the Butyl oligo-glycolate (1)

1,4,-Dioxane-2,5-dione (5.0 g, 43.1 mmol), 1-pentanol (2.2 g, 28.7 mmol) and 0.1 g tin octanoate were charged in a 20 mL reactor and heated to 120° C. for 24 hours with stirring. The resultant solution was poured into CHCl₃ and filtered through a bed of SiO₂ eluting with CHCl₃/isopropanol. The resulting mixture of oligomers was collected and dried in vacuo (3.4 g).

### Synthesis of succinic acid mono-[1-methyl-2-(2-methyl-acryloylamino)-ethyl]ester (2)

2-hydroxypropyl methacrylamide (2 g, 14 mmol) and succinic anhydride (2.10 g, 21 mmol) were dissolved in 10 mL CHCl₃. Triethyl amine (2.82 g, 28 mmol) and 4-dimethylaminopropylamine (DMAP) (170 mg, 1.3 mmol) was added along with 3 mL of DMF. The reaction turned purple. The materials were then washed with 1M HCl and a concentrated brine solution (3×50 mL). The organic layer was filtered through a silica plug eluting first with CHCl₃ and then with CHCl₃/methanol. The solvent was removed under vacuum yielding a white solid (2.2 g, 10 mmol, 65%).

Coupling of HPMA and (2) to Form the Degradable HPMA Monomer Terminated in a Butyl Group (2):

Succinic acid mono-[1-methyl-2-(2-methyl-acryloylamino)-ethyl]ester (2) (1.2 g, 4.9 mmol) was dissolved in 4 mL CHCl₃, which had been dried over 4 Å molecular sieves. To this solution was added N,N′-carbonyl diimidazole (CDI) (0.72 g, 4.4 mmol). The reaction was accompanied by bubbling and release of CO₂. After stirring for 5 hours under a nitrogen gas atmosphere, 2-hydroxypropyl methacrylate (HPMA) was added (2.65 g, ˜5 mmol) and the reaction was allowed to stir overnight. The resulting material was extracted three times with pH 5 phosphate buffer to remove any unreacted acid and imidazole. The organic layer was concentrated and the material was filtered through a silica plug eluting with CHCl₃ and then methanol. The resulting product (3) was collected and utilized as a monomer for the following step.

Co-Polymerization of 3 with HPMA to Form Degradable Polymer Component 1:

Compound 3 (0.3 g, ˜0.6 mmol) was dissolved in dioxane along with HPMA (1.17 g, 8.18 mmol) AIBN (14 mg, 87 mmol) was added. The polymer was placed in a sealed container and was degassed with nitrogen and bath sonication for 10 minutes. The material was placed under a nitrogen atmosphere and was heated to 70 C overnight (16 hours). After which no remaining monomer was detected by TLC. The solvent was removed in vacuo and the result in polymer was dissolved in DI water pH 4 (10 mM Acetate Buffer). This material was purified by SEC on Sephadex and lyophilized (1.4 g of polymer). This material functions as FIG. 5A.

Co-Polymerization of HPMA with Methacrylic Acid to Form Polymer Component 2.

HPMA (3.0 g, 21 mmol) and methacrylic acid (770 mg, 8,2 mmol) (free of inhibitor) was polymerized at 70° C. in n-propanol for 18 hours in the presence of AIBN (490 mg, 3 mmol). The material was degasses as above. The resulting product was isolated in water and the pH was transferred with 1 m NaOH to pH=4.5. This polymer was purified on Sephadex G-20 in water.

Rheological Properties after Mixing

Table 1 illustrates the change in rheological properties of the individual components of a polymer system, as well as the resulting polymer system. This was performed by making a 10 w/v solution of polymer and measuring the rheological properties of each polymer component 1 and 2 separately. These were then mixed together in the presence of 10 mm Ca²⁺ as a crosslinking agent. When the two components were mixed the polymer system formed a higher viscosity gel by entangled polymer micelles and Ca²⁺ crosslinks. The rheological properties of each component are below in Table 1 shown both alone and together:

| | Viscosity (PaS) at 1 |
|---|---|
| | sec-1 |
| Component 1 10% w/v | 32.8 |
| Component 2 10% w/v | 43.9 |
| Mixture of 1 and 2 10% w/v | 854 |
| Polymer with 10 mm Ca2+ | |

Degradation in pH 7 Over 4 Hours:

After the gel had formed, the material was vigorously mixed with pH 7.4 TRIS buffer and its viscosity was measured on a TA-instruments rheometer versus time at a sheer rate 1 s⁻¹. Stress-strain data was collected for 5 minutes and then the sample was allowed to sit undisturbed between time points. The sample showed a significant amount of degradation after 3.5 hours. As is illustrated in Table 2, the viscosity of the gel decreased over time due to hydrolytic degradation of the glycolate esters in polymer component 1 at pH 7.4.

| Time | Viscosity |
|---|---|
| (hours) | (PaS) |
| 0 | 843 |
| 0.5 | 734 |
| 1.08 | 531 |
| 1.7 | 437 |
| 2.05 | 329 |
| 2.48 | 267 |
| 3.05 | 135 |
| 3.5 | 87 |

### Example 2

### Preparation of an Ejaculate pH Induced Physically Degradable Polymers Poly(NiPAAm-co-AA-co-BMA) 80/10/10

In this example an exemplary physically degradable polymer system was produced. Here a thermogelling monomer N-isopropyl acrylamide was copolymerized with butyl methacrylate and acrylic acid. At pH 4 at room temperature the polymer system was a liquid. At body temperature and at pH 4 the system gelled by a thermogelling mechanism. When the polymer was subjected to an ejaculate at pH 7 the polymer underwent a gel to sol transition.

The synthesis of a linear terpolymers of N-isopropyl acrylamide (NiPAAm), acrylic acid (AA), and n-butyl methacrylated (BMA) with a NiPAAm/AA/BMA feed molar ratio of 80/10/10, was carried out in toluene utilizing AIBN as a free radical initiator (0.007 Eq per total monomers). The solution was then polymerized for 24 hours at 62° C. under N₂ atmosphere using a J-Kem Scientific Vortex Mixer at 50% frequency and a power level of 35. The remaining toluene was removed and the samples were dried for 24 hours under high vacuum. The dry polymer was ground down into a fine powder and triturated using dry diethyl ether. The samples were then placed in the high vacuum for 1 hour. A 2% solution of the polymer was dissolved in pH 4.2, 20 mM acetic acid buffer. The solution was filtered to remove the insoluble, very high MW polymer and then lyophilized until the sample was completely dry.

Rheological Characterization of Ejaculate pH Induced Physically Degradable Polymers NiPAAm/AA/BMA 80/10/10.

The complex viscosity of a 6% solution of NiPAAm/AA/BMA 80/10/10 polymer diluted using vaginal fluid stimulant and semen stimulant was measured. The sample was placed on a TA instruments AR550 at 37° C. and the complex viscosity was measured at an oscillatory stress of 0.64 Pa and a frequency of 1 Hz. The polymer alone at pH 4 was approximately 22.5 Pa Sec. When mixed 1:1 with vaginal fluid the complex viscosity was 86.5 Pa Sec at pH 4.3. A sample was then mixed 1:1 with semen stimulant and the complex viscosity was 2.5 Pa Sec at pH 7.4. These results showed that the polymer was a gel at the pH of the vagina and liquefied upon exposure to semen stimulant due to the change in pH upon exposure to an ejaculate.

### Example 3

### Synthesis of Thermosensitive and pH-Sensitive Linear Poly[NiPAAM-co-sulfoethyl methacrylate-co-methacrylic butyl glycolate ester)]

In this example a chemically degradable polymer system is displayed. Here a thermogelling monomer N-isopropyl acrylamide was copolymerized with the degradable sequence containing monomer methacrylic butyl glycolate ester and with sulfoethyl methacrylate to form a thermogelling and degradable ter-polymer system. At pH 4 at room temperature the polymer system is a liquid. At body temperature and at pH 4 the system gels by a thermogelling mechanism. When the polymer is subjected to an ejaculate at pH 7 the polymer undergoes a gel to sol transition due to a disruption in the thermogel structure.

### Synthesis of Butyl Glycolate (BG)

The reaction was performed in a melt of glycolide using 1.5 equivalents of glycolide with 1 eq. of butanol in the presence of 0.001 Eq of tin catalyst (Tin II ethylhexanoate 90% in hexanoic acid). The reactants were then charged into a vial/round bottom reactor containing a stir bar. The reaction vessel was then sealed and flushed with Nitrogen before being dipped in an oil bath maintained at a temperature around 135° C. The reaction was then allowed to run overnight with constant stirring. The next day, the flask was removed from the oil bath and 2-3 ml of dry CHCl₃ was added to the reaction mixture immediately to prevent the solidification of the melt. The compound was then purified by column chromatography using a silica column and 2% isopropanol+98% CHCl₃ as the solvent system. The first two fractions contained the compound. The TLC of the fractions was done using a silica TLC plate and developed by charring with PMA. The solvent was then stripped off from the combined fractions 2 and 3 and the compound was dried in high vacuum overnight. The structure was analyzed by proton NMR and C¹³ NMR.

### Synthesis of methacrylic-(butyl glycolate) ester (MGB)

The esterification of methacrylic acid with BG was done by carbonyl diimidazole coupling. 1 Eq. of methacrylic acid was charged into suitable sized round bottom flask (RBF) with a stir bar. 10 volumes of dichloromethane was then added to it. RBF was then sealed with a rubber septa and the mixture of methacrylic acid and dichloromethane was then flushed with N₂ for 5 minutes. The RBF was then placed in an ice bath until the contents cooled down to 0° C. Then CDI was then added to the reaction through the mouth of the RBF by removing the septa. Frothing was observed in the reactor. Once the frothing stopped, the reaction vessel was sealed by rubber septa and butyl glycolate was added using a syringe. The ice bath was removed and the reaction allowed to run at room temperature. It was followed by thin layer chromatography (TLC) on silica using 2% isopropanol/98% chloroform and separately using chloroform/methanol/acetic acid (CMA) 98:2:2. No spot for carbonyl diimidazole was observed after 2.5 hrs. The spot for the compound overlaps with that of carbonyl diimidazole in the TLC done using 2% isopropanol, but a distinct spot was seen for the compound in the TLC done with CMA. Once the reaction was complete, the solvent was removed in vacuo and the sample was purified by column chromatography. The yield was approximately 20%.

Free Radical Polymerization to Make the Linear Terpolymer of N-isopropylacrylamide (NiPAAM),

Methacrylic-butyl glycolate (MBG) and sulfoethyl methacrylate (SEM). 17 Eq of NiPAAM with 2 Eq of MBG, 1 Eq of SEM, 0.14 Eq of AIBN (initiator) were charged to a sealed reaction vessel with toluene as a solvent. The reaction mixture was flushed with N₂ for 10 minutes. Polymerization was then carried out in an oil bath at a temperature of 65° C. The content of the reactor solidified in an hour indicating polymerization. The white solidified polymer in toluene was iridescent when kept in the freezer for 10 minutes but turned brownish when heated to room temperature. The solvent was stripped off using rotovap and further dried in high vacuum overnight. A white flaky polymer was obtained and triturated with ethyl ether to remove any remaining monomers before being dried under vacuum overnight.

Degradation of the Linear Terpolymer of N-isopropylacrylamide (NiPAAM), methacrylic-butyl Glycolate (MBG) and Sulfoethyl Methacrylate (SEM)

A 6% solution of the polymer system was made in 4 mL vials containing solutions at pH 5, pH 7 and pH. 12. The samples at pH 4 and pH 7 gelled as the temperature was increased to 37° C. The vials were then shaken in a 37° C. bath for 1 day. The sample at pH 4 retained its viscosity whereas the sample at pH 7 experienced a decrease in viscosity. The sample at pH 12 was completely liquefied. Later NMR study was done to confirm the degradation. Six 6% solutions of the polymer were made in 20 mM pH 4.2 sodium acetate buffer. The pHs of two vials were increased using 1M NaOH solution to pH 7 and pH of two other vials were increased to pH 12. One set of vials of pH 4.2, pH 7 and pH 12 were frozen immediately in liquid N₂ and lyophilized. The other set of pH 4.2, pH 7 and pH 12 samples were kept in incubator set at 37° C. for 24 hrs. The next day the samples were frozen in liquid N₂ and lyophilized. The samples were dissolved in DMSO and proton NMR was done on 500 MHz NMR machine. The peaks corresponding to the methylene group next to the carboxyl groups of glycolide (shifts of 4.75 and 4.9) were lost in the pH 7 sample over 24 hrs. The results indicate increased degradation over time upon exposure of the polymer system to an ejaculate.

### Example 4

### Preparation of a PSA Degradable Hydrogel

In this example an exemplary enzymatically degradable polymer system was produced. A hydrogel was synthesized by creating a diamino-crosslinker containing PSA degradable sequences. The crosslinker was then reacted with preformed chains of amine reactive HPMA to form a weakly crosslinked hydrogel structure. When the polymer was subjected to an ejaculate at pH 7 containing the active seminal protease PSA the crosslinks were hydrolyzed and the gel was degraded.

### Preparation of poly(hydroxypropylmethacrylate-nitrophenylcarbonate) (pHPMA-NPC)

pHPMA-NPC was synthesized by following steps. pHPMA 0.273 g (1.9 mmol, 1 eq.) was added and dissolved in 3 mL of dry DMF in 10 mL round bottom flask. Pyridin 0.218 mL (2.7 mmol, 1.4 eq.) and catalytic amount of DMAP were added into the flask. The flask was placed and stirred in the ice bath. Nitrophenylchloroformate (NPCF) 0.5 g (2.5 mmol, 1.3 eq.) was then added into the flask. The reaction mixture was stirred in the ice for 3 hr and then at room temperature for overnight. The reaction mixture was later precipitated in the ether:acetone (2:1 v/v) mixture the next morning and dissolved in 3 mL of MeOH again. The recrystallization step was performed with the same ether/acetone solvent system and vacuum dried overnight. 0.331 g of the product was obtained and NMR analysis appeared to show approximately 10% of the hydroxyl group of pHPMA had reacted with NPCF to form a nitrophenylcarbonate group.

### Synthesis of Tetrapeptide NH2-Pro-Phe-Arg-Gly-OH

Tetrapeptide Fmoc-NH-Pro-Phe-Arg-Gly-CO₂H was synthesized on solid-phase. Wang resin 1 g (0.93 mmol reactive end, 1 eq.) was placed in the 25 mL column and rinsed with DMF 3 times. Fmoc-Gly-OH 0.829 g (2.8 mmol, 3 eq.), pyridine 0.2275 mL (2.8 mmol, 3 eq.) and diisopropylcarbodiimide (DIC) 0.352 g (2.8 mmol, 3 eq.) were dissolved in 20 mL of DMF. The solution was added into the column and shaken with Wang resin for 2 hr on wrist action shaker at room temperature. Small amount of resin was taken from the column and a Kaiser test was performed. After a negative result, the resin was treated with piperidine (20% (v/v) in DMF) for 10 min. The Kaiser test showed a positive result. After washing the resin 5 times with DMF, Fmoc-Arg-OH 1.106 g (2.8 mmol, 3 eq.), HOBT 0.427 g (2.8 mmol, 3 eq.) and DIC 0.352 g (2.8 mmol, 3 eq.) solution in DMF 20 mL was added in the column and shaken for overnight. After following the same Kaiser tests and Fmoc deprotection step, phenylalanine (3 eq.) and proline (3 eq.) addition steps were performed under same conditions as followed in arginine addition. 50% TFA in DCM (v/v) treatment was followed to cleave the peptide from the resin. The acid solvent was evaporated by rotovap and the product was additionally dried overnight under vacuum.

### Synthesis of PEG-Peptide Crosslinker

Fmoc-PFRG-OH 700 mg (1.0 mmol, 1 eq.) was added in the 22 mL vial using transfer pipette and dissolved in DCM 1.6 mL. PEG 3400 dissolved in dry DCM (1 g/2 mL) 1.4 mL (0.2 mmol, 0.2 eq.), catalytic amount of DMAP and DIC 188 uL (1.2 mmol, 1.2 eq.) were added and mixed in the solution. The reaction mixture was shaken overnight at 40° C. The product was precipitated and reprecipitated in ether and dried under high vacuum overnight. 0.395 g of the product was obtained. The mass spectrometry result showed that 50% of the hydroxyl group of the PEG was reacted with the peptide. 0.202 g (0.046 mmol, 1 eq.) of the product was additionally reacted with Fmoc-Gly-OH 0.275 g (0.9 mmol, 20 eq.), DIC 0.129 g (1.0 mmol, 22 eq.) and catalytic amount of 4-dimethyl aminopyridine in 1 mL of DCM to introduce amine groups at the unreacted end of the PEG. The reaction mixture was shaken overnight at 40° C.

### Synthesis of the PSA Degradable Gel

pHPMA-NPC 10 mg and PEG-peptide crosslinker 10 mg was dissolved in 50 uL of DMF each separately and mixed in glass vial. The mixture becomes a gel after 8 hours at room temperature. The resulting gel was washed with 3×200 μL DMF. The gel was placed in 100 mM bicarbonate buffer for 8 hours on a shaker table to hydrolyze unreacted nitrophenyl carbonate groups. The gel was then incubated for 3 days in PBS with buffer changes every 1 day.

Degradation of the Gel by Human Seminal Fluid.

Human ejaculate was collected from a healthy male and immediately placed on dry ice. The sample was then thawed in an ice bath and centrifuged at 4000 RCF for 10 minutes at 4° C. The upper plasma was separated from the sperm fraction and stored at −78° C. for further studies. The gel sample produced above was cut into small fragments (˜200 μm in diameter) and incubated in seminal fluid for 1 day. The diameter of the gel was then evaluated by microscopy. The crosslinks in the gel cross sectional area increased by 30% over a 24 hour period as the gel was degraded by the protease in the seminal fluid. Gel samples incubated in 3 fresh aliquots of seminal fluid every 24 hours completely degraded in 3 days.

The compositions and methods disclosed and claimed herein can be made and executed without undue experimentation in light of the present disclosure. While the compositions and methods of this invention have been described in terms of particular embodiments, it will be apparent to those of skill in the art that variations may be applied to the compositions and methods and/or in the steps or in the sequence of the methods described herein without departing from the concept, spirit and scope of the invention. More specifically, it will be apparent that certain related reagents may be substituted for the reagents described herein while the same or similar results would be achieved. All such substitutes and modifications apparent to those skilled in the art are deemed to be within the spirit, scope and concept of the invention as defined by the appended claims.

1. A composition comprising; a polymer system susceptible to degradation upon exposure to an ejaculate.

2. The composition of claim 1, wherein said polymer system comprises a degradable sequence.

3. The composition of claim 2, wherein said degradable sequence is susceptible to degradation of the type selected from the group consisting of chemical, physical and enzymatic.

4. The composition of claim 3, wherein said chemical degradation is caused by an agent selected from the group consisting of esters, acids, bases, alcohols and chelating agents.

5. The composition of claim 3, wherein said physical degradation is caused by a change selected from the group consisting of pH, ionic strength, temperature, pressure and sheer stress.

6. The composition of claim 3, wherein said enzymatic degradation is caused by an enzyme selected from the group consisting of proteolytic enzymes, non-proteolytic enzymes and hydrolytic enzymes.

7. The composition of claim 6, wherein said proteolytic enzymes are selected from the group consisting of peptidases, hyaluronidases, alpha glucosidases, beta glucosidases, lysophospholipases, lysozymes, mannosidases, pepsinogen I, pepsinogen II, pepsinogen III and phospholipase.

8. The composition of claim 1, wherein said degradation results in a decrease in viscosity of said polymer system.

9. The composition of claim 8, wherein said decrease in viscosity results in a gel to sol transition.

10. The composition of claim 1, wherein said polymer system includes polymers selected from the group consisting of monomer filaments, polymer filaments, modified polymers, water soluble synthetic polymers, water soluble natural polymers, acrylate based polymers, co-polymers, block co-polymers, hydrophobic degradable polymers, oligomers of hydrophobic degradable polymers and self-assembling amphiphilic monomers.

11. The composition of claim 1, further comprising a polymer backbone comprising mucoadhesive moieties.

12. The composition of claim 1, wherein said polymer system includes triblock copolymers of polyethylene oxide and polypropylene oxide polymerized with oligomeric or polymeric moieties containing anioinic groups with a pKa between 3 and 8.

13. The composition of claim 1, wherein said degradation occurs within seconds to days.

14. The composition of claim 1, further comprising microbicides.

15. The composition of claim 14, wherein said microbicides are released from said polymer system upon exposure to an ejaculate.

16. The composition of claim 14, wherein said microbicides are selected from the group consisting of entry inhibitors, fusion inhibitors, non-nucleoside reverse transcriptase inhibitors, nucleoside reverse transcriptase inhibitors, protease inhibitors, detergents, surfactants, spermicides, inhibitors of viral adsorption, inhibitors of viral proteases, antivirals, antibiotics, antifungals, anti-inflammatories, antivirals, antiparasitics, chemotherapeutics, antitoxins, immunotherapeutics, integrase inhibitors, birth control agents, fertility agents and hormone replacement agents.

17. A method comprising; administering to an individual a polymer system susceptible to degradation upon exposure to an ejaculate.

18. The method of claim 17, wherein said polymer system comprises a degradable sequence.

19. The method of claim 17, further comprising microbicides.

20. The method of claim 17, wherein said administration is for a purpose selected from the group consisting of prevention of fertility, promotion of fertility, prevention of a sexually transmitted disease, treatment of a sexually transmitted disease and hormone replacement.

21. A method for delivering microbicides to an individual upon exposure to an ejaculate, said method comprising;
(a) providing a polymer system susceptible to degradation upon exposure to an ejaculate, wherein said polymer system contains microbicides to be released upon degradation, and
(b) contacting said polymer system to a cavity selected from the group consisting of the oral, vaginal and anal cavities.

22. The method of claim 21, wherein said polymer system comprises a degradable sequence.

23. A composition comprising;
a polymer system wherein upon application to a cavity selected from the group consisting of oral, vaginal and anal cavities, the composition forms a gel susceptible to degradation upon exposure to an ejaculate.

24. A polymer system comprising;
two polymers wherein one polymer contains α-hydroxy acids susceptible to degradation upon an increase in pH upon exposure to an ejaculate wherein said polymer system experiences a reduction in viscosity in response to said increase in pH.

25. A polymer system comprising;
one or more polymers sensitive to alterations in pH wherein at an average pH found in the vaginal cavity said polymer system is a gel and wherein at an average pH found in the presence of an ejaculate, said polymer system experiences a reduction in viscosity.

26. A polymer system comprising;
one or more polymers connected by crosslinking wherein said crosslinking is susceptible to degradation upon exposure to an ejaculate.

27. The polymer system of claim 26, wherein said degradation is due to Prostate Specific Antigen in said ejaculate.

28. A composition comprising;
a polymer system susceptible to degradation by an ejaculate, said degradation of the type selected from the group consisting of chemical, physical and enzymatic.

29. The composition of claim 28, further comprising degradable sequences.

30. The composition of claim 28, wherein said polymer system further comprises microbicides.
